(19) United States
(12) Patent Application Publication
(10) Pub. No.: US 2004/0146993 A1
(43) Pub. Date: Jul. 29, 2004

(54) Isolating chondroitin sulfate

(76) Inventors: Anil B. Khare, Crystal, MN (US); Stephen Andrew Houliston, Ottumwa, IA (US); Timothy James Black, Oskaloosa, IA (US)

Correspondence Address: Edward Levine, Esq., Cargill, Incorporated (Nutraceuticals), P.O. Box 5624, Minneapolis, MN, 55440-5624, US

(21) Appl. No.: 10/704,866

(22) Filed: Nov. 10, 2003

Related U.S. Application Data
(60) Provisional application No. 60/425,809, filed on Nov. 13, 2002.

(51) Int. Cl. C12P 19/30; A61K 31/737

(52) U.S. Cl. 435/89; 514/54; 536/54

(57) ABSTRACT

A method of isolating chondroitin sulfate product from a feedstock comprising connective tissue.

### RELATED APPLICATIONS

This application claims the benefit of prior U.S. Provisional application 60/425,809, which was filed on Nov. 13, 2002, under 35 USC § 1.53(b).

### TECHNICAL FIELD

This invention relates to separation processes, and more particularly to a process for obtaining and isolating chondroitin sulfate.

### BACKGROUND

Chondroitin sulfate is a very useful glycosaminoglycan—GAG (mucopolysaccharide) and plays a key function in the metabolic transformation to larger molecules that make up proteoglycans (large molecules built from GAG). Proteoglycans are present and most abundant in connective tissues such as cartilage, tendons, skin, blood vessel walls, sclera, cornea, and intervertebral discs.

Chondroitin sulfate has been consumed as a dietary supplement for prevention and recently, treatment, of connective tissue-related ailments. It has been suggested that chondroitin can be used as an alternative treatment for osteoarthritis or degenerative joint diseases and is presently believed to aid in producing healthy connective tissue.

### SUMMARY

Certain embodiments of the invention provide a method of isolating chondroitin sulfate from feedstock that includes using a precipitant, or reagent, that removes impurities from a digested feedstock liquid. Passing the liquid digest through a membrane retains chondroitin sulfate in a retentate.

In an aspect of the invention, a process of obtaining chondroitin sulfate from a feedstock is provided, that includes supplying a feedstock that contains connective tissue; digesting the feedstock with a protease to form a liquefied digest and undigested matter; treating the liquefied digest by raising the pH to greater than about 10 with a reagent comprising a divalent hydroxide of an alkaline earth metal, to precipitate protein impurities; separating the precipitate from the treated liquefied digest; and processing the treated liquefied digest using a membrane to form a permeate and a retentate, wherein the retentate comprises chondroitin sulfate.

Advantageously, exemplary methods of the invention can be practiced without the use of added ethanol, and can process feedstock from a broad range of sources. High levels of chondroitin sulfate purity can be achieved.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. provides a flowchart depicting steps of an embodiment of the invention.

### DETAILED DESCRIPTION

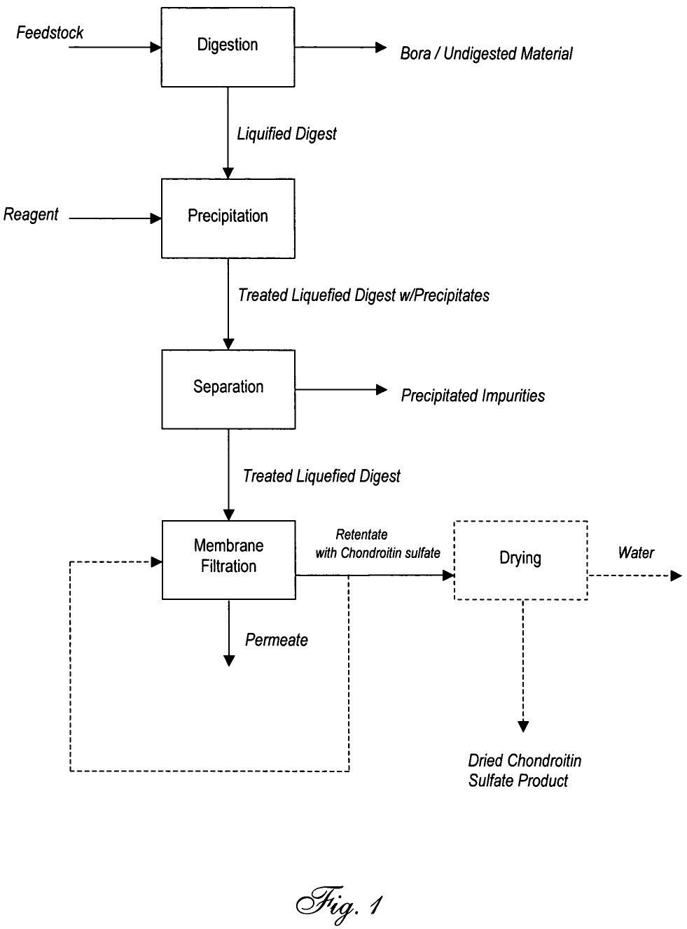

FIG. 1 is a flowchart that provides the general steps included in an exemplary method of the invention, where chondroitin sulfate is separated and obtained from a processed feedstock. As seen in the figure, chondroitin sulfate can be isolated by a process that includes: digesting a feedstock into a liquefied digest, precipitating impurities from the liquefied digest, separating the precipitate from the liquefied digest, filtering the liquefied digest through a membrane to obtain a retentate, and optionally drying the retentate to obtain chondroitin sulfate product.

In the digestion portion of the process, a feedstock is supplied to the process. The feedstock is mixed into a buffer solution having a pH of about 4 to about 7, preferably about 4.5 to about 5.5. The temperature of the feedstock/buffer mixture during digestion can be at about 55° C. to about 80° C. In an aspect of the invention, the mixture can be at about 65° C. to about 75° C. A protease can be added to the mixture to assist in digesting the feedstock. Suitable protease include for example, papain, trypsin, chymotrypsin, alkaline proteolytic enzymes, and combinations thereof. The pH of the mixture during the digestion step can be maintained at a level that avoids ill effects on the enzyme activity. Cations such as sodium acetate buffer can optionally be added to the mixture to also aid in the digestive process.

Feedstock supply for a process of the invention can include connective tissue from a variety of vertebrae. For example, suitable feedstock can be obtained from bovine, ovine, swine, equine, bird, and fish. Connective tissue such as cartilage can be useful, as nearly all cartilaginous sources found in bovine, swine, and poultry species include obtainable amounts of chondroitin sulfate. Various parts of the vertebrae that include some form of cartilage can be used, such as that from shoulder blades, navels, tracheas, gullets, etc. Feedstock need not be of high grade or “cleanliness.” Sufficient quality of chondroitin sulfate can be obtained even when feedstock includes material that has not been pre-trimmed (extraneous material such as fat is removed). Thus, a feedstock containing a broad range of parts can be supplied to a process according to the invention and still provide a high purity chondroitin sulfate product.

Digesting the feedstock produces two portions: bora (undigested material) and liquefied digest. The liquefied digest proceeds to a treatment step to remove and/or settle out undesired impurities. These can include any protein or protein degradation products, such as, but are not limited to peptides, polypeptides, amino acids, lipoproteins, sugars and the like. Extraction of the impurities can be accomplished by raising the pH using a reagent that precipitates and removes the impurities from the liquefied digest. In an aspect of the method, a reagent is added to the liquefied digest to elevate the pH to greater than about 10. A pH of about 11.0 to about 11.3 can settle lipoproteins from a liquefied digest.

An identified impurity that can be present when a broad range of feedstock grade is used is a 44 kda impurity. It has been found that this impurity can removed by settling or precipitating it out, along with other impurities, using a reagent comprising a divalent hydroxide of an alkaline earth metal. Suitable reagents include those having strong settling properties, such as for example, calcium hydroxide and magnesium hydroxide. During the precipitation portion of the process, a combination of temperature and pH can provide beneficial conditions for removing the proteins and other impurities. For example, the liquefied digest can be treated at a temperature of about 0° C. to about 80° C. The pH can be maintained at greater than about 10. In one aspect, the pH can be about 11 to about 11.5 during the precipitation phase of the process.

When a settling procedure is implemented as the treatment step, settling time for the impurities to separate from the treated liquefied digest can vary. For example, depending on the volume processed through the system, the impurities can settle out in about 20 minutes to about 15 hours. The settling time can be decreased by optionally adding a flocculating agent to the mixture. It is presently believed that calcium hydroxide functions not only as a precipitant, but can also act as a flocculating agent.

The treated liquefied digest having the precipitates therein, is then subjected to a separation step to remove the precipitated impurities from the liquefied digest. This can be accomplished by conventional techniques such as centrifugation or filtration. Separating the precipitates from the treated liquefied digest helps to ensure that the subsequent step, membrane filtration, can run effectively and efficiently. Thus, it is preferred that a substantial portion of the precipitates are removed to thereby minimize and avoid clogging the membrane with material.

In the membrane filtration step, the treated liquefied digest is passed through a membrane to separate the materials. This can be performed using a technique that separates materials according to their molecular weight. Techniques such as diafiltration or ultrafiltration can be used, in conjunction with a membrane having a specified molecular weight cutoff. To retain chondroitin sulfate product in a retentate (i.e, the liquid retained by the membrane) and allow separation and passage of higher molecular weight material as a permeate, a membrane can have a molecular weight cutoff of about 5,000 to about 15,000. According to an aspect of the process, the membrane can have a molecular weight cutoff of about 8,000 to about 10,000.

During diafiltration, a desired level of percent solids (e.g., about 1-5%) can be maintained via the addition of water. The retentate and feed pressures can vary greatly during diafiltration. This is presently believed to be partially due to a range of % solids observed in pre-diafiltration material and the impurity profile of the material. For example, the feed temperature of the liquefied digest can be maintained at about 3° C. to about 50° C.; however, diafiltration can be conducted at feed temperatures of about 15° C. to about 40° C. Feed pressure can be about 10 to about 35 psi, while the pressure of the retentate can be at about 0 to about 25 psi.

Optionally, the membrane filtration step can be repeated to concentrate and achieve a desired purity level. Higher levels of purity can be achieved, for example, if a retentate is processed though a membrane at least two times.

After membrane filtration, the retentate, if desired, can then proceed to a drying step where water is removed via, for example, evaporation, to obtain substantially dried chondroitin sulfate. Any conventional drying techniques can used, including for example, tray drying, or drum drying. If desired, drying can be performed at elevated temperatures and pressures.

According to an exemplary method of the invention, the dried material yielded after a drying step can result in greater than 90% chondroitin sulfate. In another aspect, the dried material can have greater than about 95% chondroitin sulfate.

A further optional step that can be performed in a method of the invention is a silica gel treatment. Use a silica gel can enhance the purity of the chonodroitin sulfate product.

### EXAMPLES

### Example 1

A cartilaginous feedstock was digested in a digestion buffer (pH 4.8-5.0) that consisted of 1000 g DI H₂O, 8.86 g of 50% sodium hydroxide solution in water, and 10.91 g of glacial acetic acid. Navel cartilage (601.07 g) was added to the buffer and the cocktail temperature was increased with agitation, to 65° C. Papain enzyme (5 g) was added when the temperature reached 60° C. Digestion occurred over the subsequent 4 hours. Following digestion the resulting fat layer was siphoned from the top of the cocktail and the bora/undigested material were removed via a vacuum filtration through a #1 Whatman™ filter. The remaining post digestion liquid (1302.73 g) was sent forward to lipoprotein precipitation.

In the precipitation step, an impurity classified as lipoproteins was obtained as follows. The post digestion material was cooled to 49° C. The pH of the material was elevated to 11.3 via the addition of 50% sodium hydroxide in water. The lipoprotein layer settled overnight at about 3° C. The top clear layer was transferred via a peristaltic pump into a secondary vessel, while the liquid contained in the bottom lipoprotein layer was vacuum filtered through a #5 Whatman™ filter. The post lipoprotein settled solution (1258.98 g) was determined to contain 23.17 grams of chondroitin sulfate which equates to a 3.85% yield based on feedstock weight.

The solution was further concentrated and purified using diafiltration. The post lipoprotein removal sample was heated to 40° C. and circulated via a peristaltic pump through a Millipore Pellicon II ultra filtration unit that contained an 8,000 molecular weight cut-off membrane. Feed and retentate pressures were controlled via a retentate valve, and the retentate pressure was maintained at 10 psi with the feed pressure reaching 25 psi. Diafiltration occurred for approximately 4 hours with 1409.66 g of make-up water consumed throughout the run. The final concentrate sample (150.68 g) was determined to contain 21.70 grams of chondroitin sulfate. The permeate sample (2032.22 g) was determined to have non-detectable levels of chondroitin sulfate.

The concentrate (150.68 g) was then poured into a flat bottom Pyrex™ evaporation dish, and dried under reduced pressure and at about 70° C. with a nitrogen purge. The resulting cake (22.78 g) was determined to be 92.48% pure which equates to 21.07 grams of chondroitin sulfate. The process yield was calculated to be 90.94%.

### Example 2

The solvent free purification of chrondroitin sulfate from bovine cartilage exploits a product to impurity molecular weight difference. The removal of protein impurities is possible through diafiltration, where the impurities pass through a membrane while the product is retained. The greater the product to impurity molecular weight difference becomes, the easier purity is achieved. However, the impurities of approximately the same molecular weight as the product cannot be removed.

An impurity, identified as a 44 kda impurity was found to be present in liquefied digest. The source of the impurity was determined by comparing various feedstock, trimmed (extraneous material removed—e.g., fat) versus untrimmed. It was observed tha the impact of this impurity is dependent on feedstock source, and the cleanliness of the feedstock.

The 44 kda impurity was determined to be largely contained within the extraneous material in given feedstock, not from the cartilage itself (see Table 1).

TABLE 1

| Feedstock | %44 kda Impurity |
|---|---|
| Trimmed blade A | 0.01 |
| Trimmed blade B | 0.006 |
| Extraneous trim from sample A | 0.03 |
| Extraneous trim from sample B | 0.05 |

As seen in the data, the amount of the 44 kda impurity was found to be lower when feedstock having extraneous material such as fat was trimmed off. The extraneous trim itself, when used as a feedstock produced higher levels of 44 kda impurity.

### Example 3

It has been determined that to achieve a final product of greater than 90% purity, the 44 kda impurity should be less than 0.03%, as measured with HPLC method “Percent2M.” Since it is generally not economically feasible to properly clean all feedstock sources to this level, alternative methods were sought to achieve the high purity product. Greater than 90% purity can only be achieved when blade and trimmed navel cartilage are utilized (see Table 2).

TABLE 2

| Feedstock | %44 kda Impurity |
|---|---|
| Trimmed navel | 0.006 |
| Blade | 0.03 |
| Trachea | 0.07 |
| Gullets | 0.09 |

To remove the 44 kda impurity a secondary processing step was found to be beneficial. Following digestion, the pH of the mixture was elevated to 11-11.3 (via sodium hydroxide) to settle out a lipoprotein impurity. Use of calcium hydroxide was used to elevate the pH, instead of sodium hydroxide, the 44 kda protein level was observed to significantly decrease (see Table 3). Purity of greater than 90% can be achieved with all available feedstock sources.

TABLE 3

| | | 44 kda following | 44 kda |
|---|---|---|---|
| | 44 kda following | treatment with | following treatment |
| Feedstock | digestion | (NaOH) | with Ca(OH)₂ |
| Gullets | 0.08 | 0.06 | 0.01 |
| Trachea | 0.07 | 0.06 | 0.01 |
| Blade | 0.03 | 0.01 | 0.01 |

A number of embodiments of the invention have been described. Nevertheless, it will be understood that various modifications may be made without departing from the spirit and scope of the invention. Accordingly, other embodiments are within the scope of the following claims.

What is claimed is:

1. A method of obtaining chondroitin sulfate comprising:
a) providing a feedstock comprising connective tissue;
b) digesting the feedstock with a protease to form a liquefied digest and undigested matter;
c) treating the liquefied digest with a reagent comprising a divalent hydroxide of an alkaline earth metal at a pH greater than about 10, to form a precipitate comprising protein impurities and a treated liquefied digest;
d) separating at least a portion of the precipitate from the treated liquefied digest; and
e) processing the treated liquefied digest using a membrane to form a permeate and a retentate, wherein the retentate comprises chondroitin sulfate.

2. The method according to claim 1, further comprising drying the retentate to yield a substantially dry product comprising chondroitin sulfate.

3. The method according to claim 1, wherein the connective tissue comprises cartilage.

4. The method according to claim 1, wherein the connective tissue is tissue obtained from at least one vertebrate selected from a group consisting of bovine, ovine, swine, equine, bird, and fish.

5. The method according to claim 1, wherein the protease is selected from a group consisting of papain, trypsin, chmotrypsin, alkaline proteolytic enzyme, pronase, and combinations thereof.

6. The method according to claim 1, wherein step b) is performed at a temperature of about 55° C. to about 80° C.

7. The method according to claim 1, wherein a buffer is added during step b).

8. The method according to claim 1, wherein step c) is performed at a temperature of about 0° C. to about 80° C.

9. The method according to claim 1, further comprising adding a flocculating agent in step c).

10. The method according to claim 1, wherein the reagent in step c) is calcium hydroxide.

11. The method according to claim 1, wherein step d) is conducted at a temperature of about 10° C. to about 60° F.

12. The method according to claim 2, wherein the substantially dry product comprises at least about 90% chondroitin sulfate.

13. The method according to claim 2, wherein the substantially dry product comprises at least about 95% chondroitin sulfate.

14. The method according to claim 1, wherein the membrane has a molecular weight cutoff of about 5,000 to about 15,000.

15. The method according to claim 1, wherein the membrane has a molecular weight cutoff of about 8,000 to about 10,000.

16. A powder comprising chondroitin sulfate made from the process according to claim 2.

17. A system for obtaining chondroitin sulfate comprising:
a supply apparatus configured to provide feedstock comprising connective tissue and an amount of protease;
a digestion vessel connected to the supply apparatus, the digestion vessel configured to breakdown the feedstock into a liquefied digest and undigested matter;
a reactor connected to the digestion vessel, the reactor configured to treat the liquefied digest with a reagent comprising a divalent hydroxide of an alkaline earth metal; and
a filtration apparatus following the reactor, the apparatus comprising a membrane, and wherein the apparatus is configured to allow a permeate through the membrane and hold a retentate comprising chondroitin sulfate.

18. The system of claim 17 wherein the membrane has a molecular weight cutoff of about 5,000 to about 15,000.

19. The system of claim 17, wherein the membrane has a molecular weight cutoff of about 8,000 to about 10,000.
